(19) United States
(12) Patent Application Publication
(10) Pub. No.: US 2011/0034930 A1
(43) Pub. Date: Feb. 10, 2011

(54) DRILL BURR AND METHOD FOR PERFORMING HOLES IN SUBCHONDRAL BONE TO PROMOTE CARTILAGE REPAIR

(76) Inventors: Michael D. Buschmann, Montreal (CA); Hongmei Chen, Montreal (CA)

Correspondence Address: OGILVY RENAULT LLP, 1, Place Ville Marie, SUITE 2500, MONTREAL, QC, H3B 1R1, CA

(21) Appl. No.: 12/919,889

(22) PCT Filed: Feb. 27, 2009
(86) PCT No.: PCT/CA09/00226

§ 371 Date: Oct. 25, 2010

Related U.S. Application Data
(63) Continuation of application No. 61/032,610, filed on Feb. 29, 2008.

(51) Int. Cl. A61B 17/16

(52) U.S. Cl. 606/80

(57) ABSTRACT

A method for performing holes in subchondral bone to promote cartilage repair comprises selecting a drill burr having a drilling head and an axial stop, as a function of the distance between the tip of the drilling head and the axial stop and of a desired depth to reach a desired subchondral bone marrow compartment of a patient; drilling a hole through a base of a cartilage lesion with the drill burr to reach the desired subchondral bone marrow compartment of the patient; abutting the base of the cartilage lesion defining a periphery of the hole with the axial stop while drilling; and withdrawing the drill burr from the hole; whereby the hole has the desired depth and reaches the desired subchondral bone marrow compartment to promote cartilage repair.

### CROSS-REFERENCE TO RELATED APPLICATION

This patent application claims priority on U.S. Provisional Patent Application No. 61/032,610, filed on Feb. 29, 2008.

### FIELD OF THE APPLICATION

The present application relates to cartilage repair and, more particularly, to a surgical tool and to a method for stimulating bone marrow to promote cartilage repair.

### BACKGROUND OF THE ART

Adult articular cartilage is composed of three stratified layers with distinct morphological characteristics, namely the superficial zone, the transitional zone and the radial zone. The superficial zone includes the articulating surface and contains chondrocytes with a discoidal morphology, a tangential orientation of collagen fibrils. The transitional zone, below the superficial zone, contains chondrocytes with a rounder morphology and displays a more isotropic orientation of collagen. The bulk of adult articular cartilage lies in the deepest zone, or radial zone, named to depict the radiating pattern of vertically oriented collagen fibrils emanating from the calcified cartilage layer just below the articular cartilage. The polygonally shaped chondrocytes of the radial zone are organized in vertical columns.

Below the radial zone lies the layer of calcified cartilage interdigitating with the subchondral bone plate that contains small vascularised osteons protruding into the calcified zone. This cortical subchondral bone plate then melds with marrow-rich cancellous bone. Although the above-described general morphological characteristics of adult articular cartilage are conserved across species and between different joint surfaces, the proportion of each zone and their detailed structures vary with age, species and site.

The bone-marrow stimulation family of known surgical techniques includes Pridie drilling, abrasion arthroplasty and microfracture. These methods share the common feature of intentionally injuring subchondral bone below the cartilage lesion in order to induce wound repair and tissue regrowth. Animal studies in multiple species have clearly demonstrated the intrinsic ability of injured subchondral bone to repair itself and to generate chondral repair tissue, albeit a tissue lacking hyaline articular structure and with limited reproducibility.

A randomized comparative clinical study found that microfracture was superior to autologous chondrocyte implantation (ACI) in terms of subjective clinical outcomes at two years post-treatment, and that biopsy histological appearances were similar in the two groups. A recent mixed retrospective/prospective study using MRI to compare five-year outcomes of ACI and microfracture found that, while microfracture led to slightly less lesion filling with uncharacterized tissue, it was associated with a much lower rate of reoperation compared to ACI (10% vs. 60%). Given this low level of morbidity of microfracture and an acceptable level of clinical success, microfracture remains a primary choice in many treatment algorithms for lesions of limited size (less than 2 cm²).

Unfortunately, historical widespread and nonstandardized use of microfracture has resulted in uncontrolled and inconsistent surgical technique, follow-up measures and physiotherapy programs, and consequently there remains a lack of understanding as to why microfracture appears successful for some patients and surgeons, and not for others. In addition, despite intrinsic differences between microfracture and the older and less favoured methods of Pridie drilling and abrasion arthroplasty, there have been no controlled animal studies, or clinical studies directly comparing these approaches to identify which features or consequences of these different methods influence their success. Bone-marrow stimulation procedures are widely practiced in North America, but with little scientific evidence to guide their proper implementation. There is a general lack of understanding of mechanisms and parameters that control success versus failure of bone-marrow stimulation methods for cartilage repair.

Animal studies of spontaneous repair of osteo-chondral lesions have determined that the manner in which the cartilage lesion is surgically prepared can greatly influence the repair response. Skeletally mature animals must be used in these studies, since bone-marrow-derived repair is clearly much more efficacious in young animals than in older ones. Detailed studies in an abraded equine model (46) and in drilled rabbit trochlea have identified the following sequence of events in the reparative process: hematoma formation in the subchondral space, proliferation and migration of inflammatory and stromal cells from the cancellous marrow into the fibrin clot, transformation of the fibrin clot into a vascularised provisional and cellular granulation tissue, bone remodelling, and frequently the induction of growth plate-like structures, or chondrogenic foci, within granulation tissue. These latter structures then grow in a manner similar to that seen in cartilage development where zones of proliferation, hypertrophy, calcification, vascular invasion and endochondral bone formation can be identified.

The above processes achieve variable levels of success in cartilage repair, in part due to variations in several surgically determined factors that can critically influence the success of bone-marrow stimulation procedures including: 1) size of the lesion; 2) depth of the lesion and damage to viable subchondral bone; 3) presence of the calcified cartilage layer; 4) the number of channels accessing deep marrow; 5) post-operative articulation and load-bearing. Location of the lesion can also influence success. Calcified cartilage appears to be a very effective barrier to marrow-derived repair according to several studies and species. Although removal of all calcified cartilage from cartilage lesions may maximize spontaneous repair, evidence also suggests that excessive debridement which impinges too deep into subchondral bone can result in lack of repair, subchondral cysts and ultimately, poor clinical outcome, as in abrasion arthroplasty that was performed too aggressively.

### SUMMARY OF THE APPLICATION

It is therefore an aim of the present application to provide a novel method and a novel tool for performing holes in subchondral bone to promote cartilage repair.

Therefore, in accordance with a first embodiment, there is provided a method for performing holes in subchondral bone to promote cartilage repair comprising: selecting a drill burr having a drilling head and an axial stop, as a function of the distance between the tip of the drilling head and the axial stop and of a desired depth to reach a desired subchondral bone marrow compartment of a patient; drilling a hole through a base of a cartilage lesion with the drill burr to reach the desired subchondral bone marrow compartment of the patient; abutting the base of the cartilage lesion defining a periphery of the hole with the axial stop while drilling; and withdrawing the drill burr from the hole; whereby the hole has the desired depth and reaches the desired subchondral bone marrow compartment to promote cartilage repair.

Further in accordance with the first embodiment, selecting the drill burr comprises identifying the desired depth as a function of pre-operative imagery.

Still further in accordance with the first embodiment, selecting the drill burr comprises identifying the desired depth by performing test holes in the bone.

Still further in accordance with the first embodiment, selecting the drill burr comprises identifying the desired depth by performing a qualitative assessment of a region of the bone to be drilled.

Still further in accordance with the first embodiment, drilling the hole comprises drilling the hole having a depth of at most 10.0 mm.

Still further in accordance with the first embodiment, drilling the hole comprises drilling the hole with a depth ranging between 2.0 and 6.0 mm.

Still further in accordance with the first embodiment, drilling the hole comprises drilling the hole having a diameter ranging between 0.5 and 4.0 mm.

Still further in accordance with the first embodiment, drilling the hole comprises drilling the hole having a diameter ranging between 0.9 and 2.0 mm

In accordance with a second embodiment, there is provided a drill burr for performing holes in subchondral bone to promote cartilage repair comprising: a drilling head; and a neck connected to the drilling head, a stop positioned at a specific axial distance from the drilling head on the neck, the specific axial distance corresponding to a desired depth between a base of debrided cartilage and a subchondral bone marrow compartment, and a connector portion adapted to connect the drill burr to a drill; whereby an abutment between the stop and the base of debrided cartilage during drilling enables a hole of the desired depth to be performed.

Further in accordance with the second embodiment, the drill burr comprises an irrigation space adjacent to the drilling head to irrigate a hole during drilling.

Still further in accordance with the second embodiment, the neck has a frusto-conical body between the drilling head and the stop, the frusto-conical body having a smaller diameter than the drilling head proximally to the drilling head to define the irrigation space between the drilling head and the neck.

Still further in accordance with the second embodiment, the drill burr comprises a cylindrical portion between a distal end of the frusto-conical body and the connector portion.

Still further in accordance with the second embodiment, the drilling head has a diameter ranging between 0.5 to 4.0 mm.

Still further in accordance with the second embodiment, the drilling head has a diameter ranging between 0.9 to 1.0 mm.

Still further in accordance with the second embodiment, the specific axial distance between the stop and a tip of the drilling head is at most 10.0 mm.

Still further in accordance with the second embodiment, the specific axial distance between the stop and a tip of the drilling head ranges between 2.0 and 6.0 mm.

Still further in accordance with the second embodiment, the stop is a ring mounted to the neck of the drill burr.

Still further in accordance with the second embodiment, the stop is a shoulder defined between a distal end of the neck and the connector portion.

### BRIEF DESCRIPTION OF THE DRAWINGS

FIG. 1 is a schematic side view of a drill burr in accordance with a first embodiment of the present application;

FIG. 2 is a schematic side view of a drill burr in accordance with a second embodiment of the present application; and

FIG. 3 is schematic view of a drilling head used with the drill burrs of FIGS. 1 and 2.

### DESCRIPTION OF THE PREFERRED EMBODIMENTS

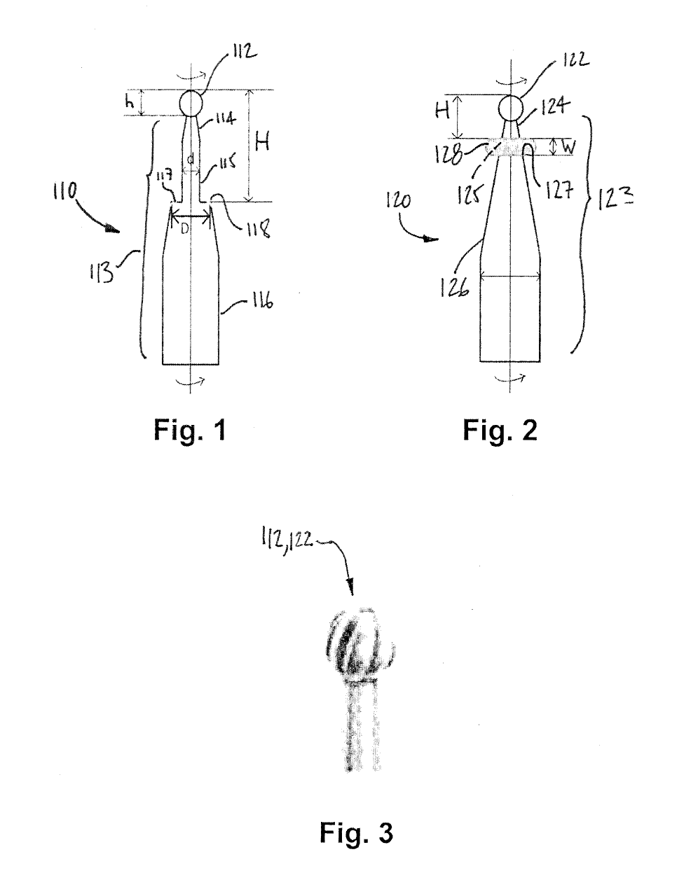

Referring to FIG. 1, a drill burr in accordance with a first embodiment is generally shown at 110. The drill burr 110 is used to drill holes through cartilage zones and calcified cartilage so as to reach the subchondral bone, to promote bone marrow stimulation for cartilage repair.

The drill burr 110 has a drilling head 112. As an example, the drilling head 112 has a diameter “h” of approximately 0.9 mm, and is shown in greater detail in FIG. 3.

The drilling head 112 is connected to the drill by way of a neck 113. The neck 113 has, amongst other parts, a frustoconical portion 114, a cylindrical portion 115, and a connector portion 116. The frusto-conical portion 114 is connected to the drilling head 112. The frustoconical portion 114 provides an irrigation space during drilling to avoid overheating the periphery of the hole, so as to reduce the risk of cell necrosis. Other configurations are considered in the neck 113 to provide irrigation during drilling.

The cylindrical portion 115 connects the frustoconical portion 114 to the connector portion 116 and has a diameter “d” that does not exceed the diameter of the drilling head 112, and preferably ranges between 0.5 mm and 0.8 mm, as an example. The connector portion 116 interfaces the drill burr 110 to the drill.

The junction between the cylindrical portion 115 and the connector portion 116 features an abutment shoulder 117 that is used as an axial stop during the drilling operation. The abutment shoulder 117 has a diameter greater than the diameter of the drilling head 112. Accordingly, when a hole is drilled using the drill burr 110, the abutment shoulder 117 abuts against the periphery of the drilled hole, so as to control the depth of the drilled hole. Therefore, the diameter “D” of the abutment shoulder 117 is greater than 0.9 mm, as an example.

The drill depth is illustrated by “H” and is the distance between the tip of the drilling head 112 and the surface of the abutment shoulder 117. Therefore, the drill depth is defined as a function of the expected depth between the exposed and debrided cartilage lesion and the desired subchondral bone marrow compartment, as it is desired to reach the deep cellular subchondral bone marrow in drilling to promote bone marrow stimulation. In one embodiment, the drill depth “H” is of 6.0 mm, but could be any other suitable value.

In order to avoid damaging the surrounding cartilage during drilling, the abutment shoulder 117 preferably has a rounded edge 118.

Referring to FIG. 2, a drill burr in accordance with another embodiment is generally shown at 120. The drill burr 120 has a drilling head 122, similar to the drilling head 112 of the drill burr 110. Therefore, the drilling head 122 has a diameter “h” of approximately 0.9 mm, for instance, and is shown in greater detail in FIG. 3. Other types of drilling heads and diameters thereof are considered as well.

The drilling head 122 is connected to the drill by the neck 123. The neck 123 has, amongst other parts, a frustoconical portion 124, a cylindrical portion 125, and a connector portion 126. The frusto-conical portion 124 is connected to the drilling head 122. Again, the frustoconical portion 124 provides an irrigation space during drilling to avoid overheating the periphery of the hole, so as to reduce the risk of cell necrosis. Other configurations are considered in the neck 123 to provide irrigation during drilling.

The cylindrical portion 125 connects the frustoconical portion 124 to the connector portion 126 and has a diameter that does not exceed the diameter of the drilling head 122. The connector portion 126 interfaces the drill burr 120 to the drill.

The junction between the cylindrical portion 125 and the connector portion 126 features an abutment shoulder 127 that supports with the cylindrical portion 125 a stopper 128. The stopper 128 is a ring that is fitted over the cylindrical portion 125 and that abuts against the abutment shoulder 127. Accordingly, the abutment shoulder 127 sets the axial position of the stopper 128 on the neck 123. By way of example, the width “W” of the stopper 128 ranges between 1.0 and 1.5 mm.

The stopper 128 is used as a stop during the drilling operation. Therefore, the stopper 128 has a diameter greater than the diameter of the drilling head 122. When a hole is drilled using the drill burr 120, the stopper 128 abuts against the periphery of the drilled hole, so as to control the depth of the drilled hole. Therefore, the diameter “D” of the stopper 128 is greater than 0.9 mm, for example.

The drill depth is illustrated by “H” and is the distance between the tip of the drilling head 112 and the exposed surface of the stopper 128. Therefore, the drill depth is defined as a function of the expected depth between the exposed cartilage and the subchondral bone, as it is desired to reach the subchondral bone in drilling to promote bone-marrow stimulation. In one embodiment, the drill depth “H” is of 2.0 mm, but could be any other suitable value.

A drill hole is preferable to a pick hole, since the drill hole does not create a compact bone interface that slows down the repair process like the pick hole may do. More important is that a deeper drill hole at a controlled depth is more effective than a shallow drill hole. Current practice does not control depth of drill holes. Thus the present application describes a drill burr with a controlled depth using abutment surfaces that are either permanent or adjustable to obtain the desired depth. The tool will provide the orthopedic surgeon with the means necessary to obtain optimal cartilage repair.

Although the preferred range of diameters for the drilling heads 112 and 122 is between 0.9 and 1.0 mm, it is considered to drill holes having diameters between 0.5 to 4.0 mm, to appropriately promote cartilage repair. As for the desired depth of the holes, it is not more than 10.0 mm, but preferably between 2.0 and 6.0 mm.

Now that the drill burrs 110 and 120 have been detailed, a method for drilling holes in bones to promote bone marrow stimulation, for instance using the drill burrs 110 or 120, is described. It is pointed out that before drilling, the cartilage lesion is first debrided with a curette or like tool using standard techniques to remove residual flaps of cartilage and the thin layer of calcified cartilage that separates noncalcified cartilage from bone.

Firstly, a drill burr, such as the drill burrs 110 or 120, is selected as a function of a desired depth of the hole to reach the cell-rich cancellous bone marrow portion of the subchondral bone of a patient. Various factors can be used to determine the desired depth, such as the type and quality of subchondral bone (dense, porous, sclerotic, etc.) and the location of the cartilage damage on the bone, as part of a qualitative assessment. Moreover, specific patient information may be taken into account in determining the desired depth, such as age and physical condition (e.g., diseases such as arthritis, etc.). In another example, the desired depth is determined by performing test holes to visually determine the suitable depth to reach the subchondral bone. Pre-poerative imaging may also be used to determine the depth of the holes. For instance, X-ray tomography may be performed pre-peratively.

Once the desired depth has been determined, and the selected drill burr has been installed on the drill, the hole is drilled through the base of the debrided cartilage lesion to reach the desired subchondral bone marrow compartment of the patient.

Because of the presence of the abutment surface 117 (FIG. 1) or of the stopper 128 (FIG. 2), the drill burr 110 or 120 (or other suitable drill burr) abuts the cartilage defining a periphery of the hole, at the desired depth. The drill burr may therefore be withdrawn from the hole, whereby the hole has the desired depth and reaches the desired subchondral bone compartment to promote cartilage repair.

It is observed that numerous holes may be performed in a damaged cartilage region, by repeating the necessary steps of the method once the drill burr has been selected and installed on the drill.

1. A method for performing holes in subchondral bone to promote cartilage repair comprising:
selecting a drill burr having a drilling head and an axial stop, as a function of the distance between the tip of the drilling head and the axial stop and of a desired depth to reach a desired subchondral bone marrow compartment of a patient;
drilling a hole through a base of a cartilage lesion with the drill burr to reach the desired subchondral bone marrow compartment of the patient;
abutting the base of the cartilage lesion defining a periphery of the hole with the axial stop while drilling; and
withdrawing the drill burr from the hole;
whereby the hole has the desired depth and reaches the desired subchondral bone marrow compartment to promote cartilage repair.

2. The method according to claim 1, wherein selecting the drill burr comprises identifying the desired depth as a function of pre-operative imagery.

3. The method according to claim 1, wherein selecting the drill burr comprises identifying the desired depth by performing test holes in the bone.

4. The method according to claim 1, wherein selecting the drill burr comprises identifying the desired depth by performing a qualitative assessment of a region of the bone to be drilled.

5. The method according to claim 1, wherein drilling the hole comprises drilling the hole having a depth of at most 10.0 mm.

6. The method according to claim 1, wherein drilling the hole comprises drilling the hole with a depth ranging between 2.0 and 6.0 mm.

7. The method according to claim 1, wherein drilling the hole comprises drilling the hole having a diameter ranging between 0.5 and 4.0 mm.

8. The method according to claim 1, wherein drilling the hole comprises drilling the hole having a diameter ranging between 0.9 and 2.0 mm.

9. A drill burr for performing holes in subchondral bone to promote cartilage repair comprising:
a drilling head; and
a neck connected to the drilling head, a stop positioned at a specific axial distance from the drilling head on the neck, the specific axial distance corresponding to a desired depth between a base of debrided cartilage and a subchondral bone marrow compartment, and a connector portion adapted to connect the drill burr to a drill;
whereby an abutment between the stop and the base of debrided cartilage during drilling enables a hole of the desired depth to be performed.

10. The drill burr according to claim 9, further comprising an irrigation space adjacent to the drilling head to irrigate a hole during drilling.

11. The drill burr according to claim 10, wherein the neck has a frusto-conical body between the drilling head and the stop, the frusto-conical body having a smaller diameter than the drilling head proximally to the drilling head to define the irrigation space between the drilling head and the neck.

12. The drill burr according to claim 11, further comprising a cylindrical portion between a distal end of the frusto-conical body and the connector portion.

13. The drill burr according to claim 9, wherein the drilling head has a diameter ranging between 0.5 to 4.0 mm.

14. The drill burr according to claim 9, wherein the drilling head has a diameter ranging between 0.9 to 1.0 mm.

15. The drill burr according to claim 9, wherein the specific axial distance between the stop and a tip of the drilling head is at most 10.0 mm.

16. The drill burr according to claim 9, wherein the specific axial distance between the stop and a tip of the drilling head ranges between 2.0 and 6.0 mm.

17. The drill burr according to claim 9, wherein the stop is a ring mounted to the neck of the drill burr.

18. The drill burr according to claim 9, wherein the stop is a shoulder defined between a distal end of the neck and the connector portion.

19. Use of a drill burr to perform holes in subchondral bone to promote cartilage repair, the drill burr comprising:
a drilling head; and
a neck connected to the drilling head, a stop positioned at a specific axial distance from the drilling head on the neck, the specific axial distance corresponding to a desired depth between a base of debrided cartilage and a subchondral bone marrow compartment, and a connector portion adapted to connect the drill burr to a drill;
whereby an abutment between the stop and the base of debrided cartilage during drilling enables a hole of the desired depth to be performed.

20. Use of the drill burr according to claim 19, the drill burr comprising an irrigation space adjacent to the drilling head to irrigate a hole during drilling.
